(19) United States
(12) Patent Application Publication
(10) Pub. No.: US 2018/0161510 A1
(43) Pub. Date: Jun. 14, 2018

(54) DRUG DELIVERY DEVICE

(71) Applicant: SANOFI-AVENTIS DEUTSCHLAND GMBH, Frankfurt am Main (DE)

(72) Inventors: James Coop, Clifton, Bristol (GB); Paul Hayton, St. Andrews, Bristol (GB); James West, Bristol (GB); Stephen Francis Gilmore, Bristol (GB)

(21) Appl. No.: 15/576,613

(22) PCT Filed: May 24, 2016
(86) PCT No.: PCT/EP2016/061651

§ 371 Date: Nov. 22, 2017

(30) Foreign Application Priority Data
May 27, 2015 (EP) 15169309.0

(51) Int. Cl. A61M 5/315; A61M 5/19

(52) U.S. Cl. CPC A61M 5/31551; A61M 5/31556; A61M 5/31536; A61M 5/19

(57) ABSTRACT

Drug delivery device comprising a housing retaining a primary drug delivery assembly for the delivery of a primary medicament with a primary dose setting member configured to rotate in a helical movement to set a dose of the primary medicament and a secondary drug delivery assembly for the delivery of a secondary medicament. A dose dial is movable between a first position and a second position in axial direction and is rotationally constrained with respect to the housing when moved from the first position into the second position and is rotationally constrained to the primary dose setting member in the second position. A dispense actuator moves in distal direction to initiate dispense of the secondary medicament. The dose dial and the dispense actuator can be coupled via a clutch component for dispense of the secondary medicament.

### CROSS REFERENCE TO RELATED APPLICATIONS

The present application is the national stage entry of International Patent Application No. PCT/EP2016/061651, filed on May 24, 2016, and claims priority to Application No. EP 15169309.0, filed in on May 27, 2015, the disclosures of which are expressly incorporated herein in entirety by reference thereto.

### TECHNICAL FIELD

The present disclosure is directed to a drug delivery device for injecting two or more medicaments from separate medicament cartridges with a primary drug delivery assembly and a secondary drug delivery assembly.

### BACKGROUND

Certain disease states require treatment using one or more different medicaments. Some drug compounds need to be delivered in a specific relationship with each other in order to deliver the optimum therapeutic dose. Here, combination therapy may be desirable, but not possible in a single formulation for reasons such as, but not limited to, stability, compromised therapeutic performance and toxicology.

For example, in some cases it might be beneficial to treat a diabetic with a long acting insulin and with a glucagon-like peptide-1 (GLP-1), which is derived from the transcription product of the proglucagon gene. GLP-1 is found in the body and is secreted by the intestinal L cell as a gut hormone. GLP-1 possesses several physiological properties that make it (and its analogs) a subject of intensive investigation as a potential treatment of diabetes mellitus. Another example of a medicament combination is the administration of a pain reliever in combination with a medicament for treating osteoarthritis. For example, a non-adjustable fixed dose of an anesthetic could be combined with an adjustable dose of an inflammatory medicament against, e.g. rheumatoid arthritis.

Drug delivery devices of the aforementioned kind often have applications where regular injection by persons without formal medical training occurs. This is increasingly common among patients having diabetes or the like, e.g. osteoarthritis. Self-treatment enables such patients to conduct effective management of their disease.

There are basically two types of drug delivery devices: resettable devices (i.e., reusable) and non-resettable (i.e., disposable). For example, disposable pen delivery devices are supplied as self-contained devices. Such self-contained devices do not have removable pre-filled cartridges. Rather, the pre-filled cartridges may not be removed and replaced from these devices without destroying the device itself. Consequently, such disposable devices need not have a resettable dose setting mechanism. The present disclosure is applicable for both types of devices, i.e. for disposable devices as well as for reusable devices.

In combination therapy, a primary medicament and a secondary medicament are delivered in a specific relationship to deliver the optimum therapeutic dose. The injection devices of the generic kind usually comprise a housing in which two or more drug delivery assemblies are retained. Such devices include a primary drug delivery assembly for dispensing the primary medicament such as the long-acting insulin and a secondary drug delivery assembly for dispensing the secondary medicament, such as GLP-1. Some kinds of drug delivery assemblies comprise a compartment such as a cartridge holder for accommodating a replaceable medicament container such as a cartridge which stores the medicament.

In some cases, depending on the patient or the stage of the therapy, an effective treatment requires variations in the quantities and/or proportions of the medicaments making up the combined therapy. For example, the patient may require a non-adjustable fixed dose of the secondary medicament in combination with an adjustable variable dose of the primary medicament.

The effectiveness of a combined delivery of medicaments may require one or more doses to be delivered sequentially with one of the two medicaments being injected into the human body prior to the delivery of the other medicament. Such treatment may be conducted with devices that include two separate dispensing mechanisms that are actuated independently from each other such that the dispensing mechanisms are activated successively. However, such devices are difficult for users to handle.

Accordingly, there still exists a strong need to provide users of such injection devices with an easy to use device that allows safer and more convenient setting of two doses of medicaments stored in separate compartments.

### SUMMARY

The present disclosure is in particular suitable for drug delivery devices configured for replaceable cartridges. For example, by detaching the cartridge holder from the housing body, the user may replace an empty cartridge with a new one. Then the cartridge holder may be reattached to the housing body. However, the disclosure also includes disposable drug delivery devices that are usually thrown away by the user after the content of medicament cartridge has been dispensed.

Drug delivery devices of the generic kind are also called dual cartridge drug delivery devices and comprise a housing retaining a primary drug delivery assembly for the delivery of a primary medicament, wherein the primary drug delivery assembly comprises a primary dose setting member such as a dose setting sleeve, configured to rotate in a helical movement in a proximal direction, usually in a first rotational direction, to set a dose of the primary medicament to be dispensed, and a secondary drug delivery assembly for the delivery of a secondary medicament. When a dose of the primary medicament has not been set, the primary dose setting member is in a zero unit dose position, the most distal position of the primary dose setting member. The disclosure includes a dose dial movable between a first position and a second position in axial direction with respect to the primary dose setting member, wherein the dose dial is configured in such way that the dose dial is rotationally constrained with respect to the housing (in the first position and) when moved from the first position into the second position when the primary dose setting member is in a zero unit dose position and in such way that the dose dial is rotationally constrained to the primary dose setting member in the second position and is free to rotate relative to the housing so that a dose of the primary medicament is set. The secondary drug delivery assembly further comprises a dispense actuator configured to move in a distal direction to initiate dispense of the secondary medicament, and a clutch component having a thread interface configured for engagement with a thread interface provided on the dose dial and coupled to the dispense actuator in such way that displacement of the clutch component in distal direction causes dispense of the secondary medicament. The drug delivery device is configured such that when the dose dial is moved from the first into the second position, the clutch component is moved into engagement with the dose dial so that during rotation of the dose dial in the second position, the thread interface of the dose dial and the clutch component engage such that the dose dial moves in axial direction relative to the clutch component when rotated and such that pure axial motion of the dose dial in distal direction is transferred to the dispense actuator.

By means of the dose dial the user can set a dose of the primary medicament to be dispensed by rotation. As the user is required to pull the dose dial axially from the first into the second position prior to the setting of a dose of the primary medicament, rotation of the primary dose setting member is prevented until the dose dial reaches the second position. Rotation in the second position sets a dose of the primary medicament with relative axial displacement between the dose dial and the clutch component without affecting the axial position of the dispense actuator. The dose dial that has been used for setting the dose of the primary medicament can therefore initiate the dispense of the secondary medicament. The engagement between the dose dial and the coupling element is coupled when the dose dial is moved from the first into the second position to prepare the device ready for setting a dose of the primary medicament. The dose dial may be permanently keyed to the primary dose setting member. The dose dial may be provided with spline features such as teeth that engage corresponding spline features on the primary dose setting member in the second position.

The housing may retain the drug deliver assemblies, with the drug delivery assemblies being placed next to each other. At a distal section, medicament cartridges with the two medicaments may be attached. The drug delivery assemblies respectively comprise a piston rod, a lead screw or the like configure to displace in distal direction during dispense such as to displace a bung in the respective medicament cartridge. A “2 to 1” needle adapter may be attached to the housing. In a preferred embodiment, this adapter is a disposable component; however, it is expediently designed so that it can be reused for multiple injections before it must be discarded. The delivery needle may be replaced after every injection. The delivery needle may attach to a hub that is integral to the adapter. The adapter includes two needles that are respectively configured for fluid communication with one of the two medicament cartridges and the injection needle.

A suitable drug delivery assembly that may be retained in the housing as the primary drug delivery assembly is described in EP 1 603 610 A1, which is incorporated herein by reference. In such device, modifications as described in the following are possible. The primary drug delivery assembly comprises a dose dial sleeve as dose setting member which can be coupled to the dose dial. The primary drug delivery assembly comprises a piston rod with a first threaded portion at a first (distal) end and a second threaded portion at a second (proximal) end. The first thread and the second thread are oppositely disposed. An insert is provided with a threaded insert, which is in threaded engagement with the first threaded portion of the piston rod. A drive sleeve extends about the piston rod. The drive sleeve is generally cylindrical. The drive sleeve is provided at a first (distal) end with a first radially extending flange. A second radially extending flange is provided spaced a distance along the drive sleeve from the first flange. An intermediate thread is provided on an outer part of the drive sleeve extending between the first flange and the second flange. A helical groove extends along the internal surface of the drive sleeve. The second thread of the piston rod is adapted to work within the helical groove.

A shoulder is formed between a proximal end of the drive sleeve and an extension provided at the proximal end of the drive sleeve. The extension has reduced inner and outer diameters in comparison to the remainder of the drive sleeve. A second end of the extension is provided with a radially outwardly directed flange.

A clutch is disposed about the drive sleeve, between the drive sleeve and the dose setting member, which is configured as a dose dial or dose setting sleeve. The dose dial sleeve rotates in a helical movement, a combination of rotation and axial displacement during dose setting. The dose dial sleeve is provided outside of the clutch and radially inward of the main housing. A helical groove is provided about an outer surface of the dose dial sleeve engaging a helical rib provided by the housing. The helical groove on the dose dial sleeve and the helical groove in the drive sleeve have the same lead. This allows the dose dial sleeve to extend from the main housing and the drive sleeve to climb the piston rod at the same rate

The clutch is cylindrical and is provided at a distal end with a series of saw teeth. Towards the proximal end of the clutch means there is located a radially inwardly directed flange. The flange of the clutch means is disposed between the shoulder of the drive sleeve and the radially inwardly directed flange of the extension. The proximal end of the clutch means is provided with a plurality of dog teeth adapted to engage with a second end of the dose dial sleeve. The clutch is keyed to the drive sleeve by way of splines to prevent relative rotation between the clutch and the drive sleeve.

A clicker spring is located adjacent the proximal flange of the drive sleeve and splined to the housing. The clicker spring has a flexible arm extending in proximal direction, engaging the saw teeth of the clutch. A dose dial grip is secured to the dose dial sleeve to prevent relative movement therebetween. The dose dial grip is provided with a central opening. A dispense button of generally ‘T’ section is provided at a second end of the pen-type injector. A stem of the button may extend through the opening in the dose dial grip.

Dose setting is performed by rotation of the dose dial which rotates the dose dial sleeve. When the desired dose has been dialed, the user may then dispense this dose by de-pressing the button. The button engages and displaces the clutch axially with respect to the dose dial sleeve causing the dog teeth to disengage. However, the clutch means remains keyed in rotation to the drive sleeve. The dose dial sleeve and associated dose dial grip are now free to rotate (guided by the helical rib located in helical groove).

The axial movement deforms the flexible arm of the clicker spring and ensures that the saw teeth cannot be overhauled during dispense. This rotationally locks the clutch to the main housing and prevents the drive sleeve from rotating with respect to the main housing though it is still free to move axially with respect thereto. This deformation is subsequently used to urge the clutch back into proximal direction to restore the connection between the clutch and the dose dial sleeve when pressure is removed from the button.

The longitudinal axial movement of the drive sleeve causes the piston rod to rotate through the opening in the insert, thereby to advance the piston in the cartridge. In the sense of the disclosure, the dial grip of the above described drug delivery assembly may form the respective dose setting member. Of course, the dose dial may also form the primary dose setting member.

The secondary drug delivery assembly may comprise a drive sleeve, herein referred to as the secondary drive sleeve and a lead screw with a first and a second threaded portion, wherein the first threaded position is in threaded engagement with a thread that is fixed in the housing of the device so that rotation of the lead screw displaces the lead screw in a distal direction. At the end of the lead screw there may be provided a bearing for displacing a bung in a medicament cartridge.

The second threaded portion is in threaded engagement with an internal thread of the secondary drive sleeve, wherein the second threaded portion is formed on flexible arms such that proximal movement of the secondary drive sleeve causes the flexible arms to deflect, preferably, inwardly and the secondary drive sleeve to move relative to the lead screw. When the drive sleeve is moved distally, this threaded engagement causes the lead screw to rotate and to displace distally because the flexible arms do not deflect.

The lead screw threads may be opposite hands. The interaction of the drive sleeve, the lead screw and an insert, which may be a lead screw nut, results in a mechanical reduction of the linear travel of the drive sleeve travel to the linear travel of the lead screw. The drive sleeve is designed such that it can travel axially with respect to the housing but cannot rotate. The thread at the proximal end of the lead screw is formed on the flexible arms and the thread-form of the drive sleeve with which it co-operates may be ramped on one side. To set a dose of the secondary medicament to be dispensed, the drive sleeve is moved axially in proximal direction relative to the housing. During setting, the flexible arms deflect so that the secondary drive sleeve moves proximally relative to the lead screw. The set dose of the secondary medicament is dispensed by moving the secondary drive sleeve axially in distal direction to return it to its starting position. By this action, the thread form of the drive sleeve engages the flexible arm of the lead screw and the lead screw is caused to rotate. Because the lead screw is threaded to the lead screw nut it advances through this thread to dispense the set dose of the secondary medicament. The lead screw advances by a distance that corresponds to the relationship between the leads of the first and second threads.

According to a further embodiment of the disclosure, the dose dial is configured as a sleeve at least partly surrounds the primary dose setting member.

The housing may retain the drug deliver assemblies, with the drug delivery assemblies being placed next to each other. At a distal section, medicament cartridges with the two medicaments may be attached

According to a further embodiment of the disclosure, the clutch component is slidably guided on the dispense actuator between a first position where the clutch component is disengaged from the dose dial and a second position where the clutch component engages the dose dial, preferably along a direction transverse to a longitudinal (axial) direction of the device. For that purpose, the clutch component may have a groove or the like engaged by a rib of the dispense actuator. The rib and the groove may be aligned in said direction transverse to said longitudinal axis of the device extending from a distal to a proximal end of the device. This has benefits, as the two drug delivery devices are arranged next to each other.

According to a further embodiment of the disclosure, the secondary drug delivery assembly comprises a secondary dose setting member configured to move in proximal direction to set a dose of the secondary medicament, wherein the dose dial is configured to engage a transfer collar when moved from the first into the second position, wherein the transfer collar transfers proximal movement of the dose dial to the secondary dose setting member such that a fixed dose of the secondary medicament is automatically set. Generally, a transfer element can be used to engage the clutch component and to move the clutch component into the second position. Also, the secondary dose setting member can be adapted such that proximal movement of the secondary dose setting member causes the secondary dose setting member to move the clutch component into the second position and into engagement with the dose dial such that the thread interface can be brought into engagement.

According to a further embodiment of the disclosure, the secondary drive sleeve is coupled to the secondary dose setting member and is configured to move proximally to set a dose of the secondary medicament and is further configured to move distally during dispense such as to drive the lead screw in distal direction. The secondary dose setting member can be integrally formed with the secondary drive sleeve. In fact, the transfer element can be adapted to push the secondary drive sleeve proximally when urged by the dose dial in proximal direction.

According to a further embodiment of the disclosure, the dispense actuator is configured to move to the secondary drive sleeve distal direction when actuated. As a result, the drive sleeve drives the lead screw such that a set dose of the secondary medicament is dispensed.

According to a further embodiment of the disclosure, the secondary drive sleeve has pins or other suitable projections engaging a (first) track on the clutch component, wherein the (first) track is configured such that movement of the pins in the (first) track causes the clutch component to engage the dose dial when a dose of the secondary medicament is set. The (first) track may be formed with slanted surfaces which guide and move the clutch component from the first position into the second position, when the pins move relative to the (first) track. When the secondary drive sleeve is moved in proximal direction to set a dose of the secondary medicament, the pins urge the clutch component such that the thread interface between the clutch component and the dose dial can be established. This interface is then used to transfer a dispensing force induced into the dose dial to the dispensing actuator through the clutch component.

According to a further embodiment of the disclosure, the pins are provided on flexible arms that are configured to deflect from the first track when the secondary drive sleeve is moved in distal direction such as to engage a second track on the clutch component wherein the second track is configured such that axial displacement of the secondary drive sleeve moves the clutch component into the first position. Accordingly, when the secondary medicament is dispensed, the thread interface engagement between the dose dial and the clutch component is removed again. To deflect the flexible arms, the flexible arms may be provided with angled surfaces that engage a fixed section of the housing when the secondary drive sleeve moves distally. The pins are redirected into the second track.

To reset the clutch component, a reset spring may be provided that is arranged such as to bias the secondary drive sleeve in axial direction after dispense of the secondary medicament.

According to a further embodiment of the disclosure, a dose button is arranged in the dose dial in such way that displacement of the dose button in distal direction causes the dose dial to move in distal direction from the second into the first position relative to the primary dose setting member such that a set dose of the secondary medicament is dispensed. To ensure that the secondary medicament is dispensed prior to the primary medicament, according to a further embodiment of the disclosure, a dose button is arranged in the dose dial in such way that displacement of the dose button in distal direction causes the dose dial to move in distal direction from the second into the first position such that a set dose of the secondary medicament is dispensed and in such way that when the dose dial is in the first position, the dose button engages a dispense button of the primary drug delivery assembly such that further movement of the dose button causes the set dose of the primary medicament to be dispensed.

Preferably, after a dose of the primary medicament has been set and when the dose dial is moved from the second into the first position relative to the primary dose setting member again, the dose dial is locked against rotation to ensure that the thread engagement between the dose dial and the clutch component transfers the axial motion of the dose dial to the dispense actuator of the secondary drug delivery assembly.

According to a further embodiment of the disclosure, the primary dose setting member is in threaded engagement with a thread provided in the housing or on an insert, which has the same lead as the thread engagement between the clutch component and the dose dial.

According to a further embodiment of the disclosure, the dose dial is axially splined to the housing in the first position and is splined to the primary dose setting member in the second position. This provides extra safety in terms of not dialing a dose of the primary medicament in the first phase of the dose setting. A flexing arm on the dose dial may engage a rib in the housing to lock the dose dial against rotation, wherein the dose dial disengages from the rib on the housing the second position.

For determining a fixed dose that cannot be influenced by the user in the combined therapy treatment, a stop may be provided to limit the axial travel of the secondary drive sleeve in proximal direction, wherein the secondary drive sleeve reaches the stop when the dose dial reaches the second position.

In order to prevent the delivery of the secondary medicament until a dose of the primary medicament has been set, a further embodiment of the disclosure comprises a locking element configured to engage the secondary drive sleeve when the dose dial is moved from the first into the second position relative to the primary dose setting member such that movement of the secondary drive sleeve in distal direction is prevented, wherein the dose dial prevents the locking element from disengaging from the secondary drive sleeve, wherein the dose dial is configured such rotation of the dose dial in the second position to set a dose of the primary medicament allows the locking element to disengage from the drive sleeve such that displacement of the drive sleeve in distal direction is allowed.

According to a further embodiment of the disclosure, the dose dial is splined to a rib provided in the housing until the dose dial has been moved from the first into the second position. This rib prevents rotation of the dose dial and consequently the setting of a dose of the primary medicament. Thus, the user actions always include to the setting of a dose of the secondary medicament. According to a further embodiment of the disclosure, the primary dose setting member is configured to move in distal direction in a helical movement during dispense of the primary medicament, wherein the dose dial is configured to reengage with the rib after dispense of the primary medicament. For this purpose, the dose dial may have a flexible element that engages the rip in a snapping action, Thereby, after dispense, the device is again in the condition which requires the user to pull the dose dial.

According to a further embodiment, the drug delivery device comprises a primary medicament cartridge and a secondary medicament cartridge, wherein the primary medicament cartridge is coupled to the primary drug delivery assembly and the secondary medicament cartridge is coupled to the secondary drug delivery assembly, and wherein the primary and the secondary medicament cartridge contain a medicament. The cartridges may be accommodated in an at least partly transparent cartridge holder.

According to a further embodiment of the disclosure, the drug delivery device is configured as a disposable device.

The primary dose setting member may be configured as a number sleeve with a dose scale provided thereon. For indicating the set doses, the number sleeve dose scale may incorporate two number rows with one number row indicating the set dose of the primary medicament and with the other number row indicating the set dose of the secondary medicament.

The device efficiently prevents mono-dosing of the primary and of the secondary medicament and ensures that the patient receives a fixed dose of the secondary medicament before the primary medicament can be administered.

The device efficiently facilitates that the medicaments are administered one after the other thus preventing mixing with one another. This ensures that each medicament provides its intended effect alone without interacting with each other.

The effect that the non-adjustable fixed dose is administered before the adjustable dose is particularly beneficial for example when a non-adjustable fixed dose of an anesthetic is administered to provide pain relief and is combined with an adjustable dose of an inflammatory medicament against, e.g. rheumatoid arthritis. Thus the painful administration of a medicament can be mitigated with administering a local anesthetic beforehand. Another example would be a combination therapy for patients suffering from diabetes that require a fixed dose of a GLP-1 and an individual dose of a long-acting insulin in a combination therapy.

According to a further embodiment of the disclosure, the drug delivery device is configured as a disposable device.

The term “medicament”, as used herein, means a pharmaceutical formulation containing at least one pharmaceutically active compound, wherein in one embodiment the pharmaceutically active compound has a molecular weight up to 1500 Da and/or is a peptide, a protein, a polysaccharide, a vaccine, a DNA, a RNA, an enzyme, an antibody or a fragment thereof, a hormone or an oligonucleotide, or a mixture of the above-mentioned pharmaceutically active compound,

wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,

wherein in a further embodiment the pharmaceutically active compound comprises at least one peptide for the treatment and/or prophylaxis of diabetes mellitus or complications associated with diabetes mellitus such as diabetic retinopathy,

wherein in a further embodiment the pharmaceutically active compound comprises at least one human insulin or a human insulin analogue or derivative, glucagon-like peptide (GLP-1) or an analogue or derivative thereof, or exendin-3 or exendin-4 or an analogue or derivative of exendin-3 or exendin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivatives are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-Y-glutamyl)-des(B30) human insulin; B29-N—(N-lithocholyl-Y-glutamyl)-des(B30) human insulin; B29-N-(ω-carboxyheptadecanoyl)-des(B30) human insulin and B29-N-(ω-carboxyheptadecanoyl) human insulin.

Exendin-4 for example means Exendin-4(1-39), a peptide of the sequence H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:

### H-(Lys)4-des Pro36, des Pro37 Exendin-4(1-39)-NH2,

### H-(Lys)5-des Pro36, des Pro37 Exendin-4(1-39)-NH2,

### des Pro36 Exendin-4(1-39),

### des Pro36 [Asp28] Exendin-4(1-39),

### des Pro36 [IsoAsp28] Exendin-4(1-39),

### des Pro36 [Met(O)14, Asp28] Exendin-4(1-39),

### des Pro36 [Met(O)14, IsoAsp28] Exendin-4(1-39),

### des Pro36 [Trp(O2)25, Asp28] Exendin-4(1-39),

### des Pro36 [Trp(O2)25, IsoAsp28] Exendin-4(1-39),

### des Pro36 [Met(O)14 Trp(O2)25, Asp28] Exendin-4(1-39),

### des Pro36 [Met(O)14 Trp(O2)25, IsoAsp28] Exendin-4(1-39); or

### des Pro36 [Asp28] Exendin-4(1-39),

### des Pro36 [IsoAsp28] Exendin-4(1-39),

### des Pro36 [Met(O)14, Asp28] Exendin-4(1-39),

### des Pro36 [Met(O)14, IsoAsp28] Exendin-4(1-39),

### des Pro36 [Trp(O2)25, Asp28] Exendin-4(1-39),

### des Pro36 [Trp(O2)25, IsoAsp28] Exendin-4(1-39),

### des Pro36 [Met(O)14 Trp(O2)25, Asp28] Exendin-4(1-39),

### des Pro36 [Met(O)14 Trp(O2)25, IsoAsp28] Exendin-4(1-39),

wherein the group -Lys6-NH2 may be bound to the C-terminus of the Exendin-4 derivative;
or an Exendin-4 derivative of the sequence

### des Pro36 Exendin-4(1-39)-Lys6-NH2 (AVE0010),

### H-(Lys)6-des Pro36 [Asp28] Exendin-4(1-39)-Lys6-NH2,

### des Asp28 Pro36, Pro37, Pro38Exendin-4(1-39)-NH2,

### H-(Lys)6-des Pro36, Pro38 [Asp28] Exendin-4(1-39)-NH2,

### H-Asn-(Glu)5des Pro36, Pro37, Pro38 [Asp28] Exendin-4(1-39)-NH2,

### des Pro36, Pro37, Pro38 [Asp28] Exendin-4(1-39)-(Lys)6-NH2,

### H-(Lys)6-des Pro36, Pro37, Pro38 [Asp28] Exendin-4(1-39)-(Lys)6-NH2,

### H-Asn-(Glu)5-des Pro36, Pro37, Pro38 [Asp28] Exendin-4(1-39)-(Lys)6-NH2,

### H-(Lys)6-des Pro36 [Trp(O2)25, Asp28] Exendin-4(1-39)-Lys6-NH2,

### H-des Asp28 Pro36, Pro37, Pro38 [Trp(O2)25] Exendin-4(1-39)-NH2,

### H-(Lys)6-des Pro36, Pro37, Pro38 [Trp(O2)25, Asp28] Exendin-4(1-39)-NH2,

### H-Asn-(Glu)5-des Pro36, Pro37, Pro38 [Trp(O2)25, Asp28] Exendin-4(1-39)-NH2,

### des Pro36, Pro37, Pro38 [Trp(O2)25, Asp28] Exendin-4(1-39)-(Lys)6-NH2,

### H-(Lys)6-des Pro36, Pro37, Pro38 [Trp(O2)25, Asp28] Exendin-4(1-39)-(Lys)6-NH2,

### H-Asn-(Glu)5-des Pro36, Pro37, Pro38 [Trp(O2)25, Asp28] Exendin-4(1-39)-(Lys)6-NH2,

### H-(Lys)6-des Pro36 [Met(O)14, Asp28] Exendin-4(1-39)-Lys6-NH2, des Met(O)14 Asp28 Pro36, Pro37, Pro38 Exendin-4(1-39)-NH2,

### H-(Lys)6-desPro36, Pro37, Pro38 [Met(O)14, Asp28] Exendin-4(1-39)-NH2,

### H-Asn-(Glu)5-des Pro36, Pro37, Pro38 [Met(O)14, Asp28] Exendin-4(1-39)-NH2, des Pro36, Pro37, Pro38 [Met(O)14, Asp28] Exendin-4(1-39)-(Lys)6-NH2,

### H-(Lys)6-des Pro36, Pro37, Pro38 [Met(O)14, Asp28] Exendin-4(1-39)-(Lys)6-NH2,

### H-Asn-(Glu)5 des Pro36, Pro37, Pro38 [Met(O)14, Asp28] Exendin-4(1-39)-(Lys)6-NH2,

### H-Lys6-des Pro36 [Met(O)14, Trp(O2)25, Asp28] Exendin-4(1-39)-Lys6-NH2,

### H-des Asp28 Pro36, Pro37, Pro38 [Met(O)14, Trp(O2)25] Exendin-4(1-39)-NH2,

### H-(Lys)6-des Pro36, Pro37, Pro38 [Met(O)14, Asp28] Exendin-4(1-39)-NH2,

### H-Asn-(Glu)5-des Pro36, Pro37, Pro38 [Met(O)14, Trp(O2)25, Asp28] Exendin-4(1-39)-NH2, des Pro36, Pro37, Pro38 [Met(O)14, Trp(O2)25, Asp28] Exendin-4(1-39)-(Lys)6-NH2,

### H-(Lys)6-des Pro36, Pro37, Pro38 [Met(O)14, Trp(O2)25, Asp28] Exendin-4(S1-39)-(Lys)6-NH2,

### H-Asn-(Glu)5-des Pro36, Pro37, Pro38 [Met(O)14, Trp(O2)25, Asp28] Exendin-4(1-39)-(Lys)6-NH2;

or a pharmaceutically acceptable salt or solvate of any one of the afore-mentioned Exendin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Antibodies are globular plasma proteins (˜150 kDa) that are also known as immunoglobulins which share a basic structure. As they have sugar chains added to amino acid residues, they are glycoproteins. The basic functional unit of each antibody is an immunoglobulin (Ig) monomer (containing only one Ig unit); secreted antibodies can also be dimeric with two Ig units as with IgA, tetrameric with four Ig units like teleost fish IgM, or pentameric with five Ig units, like mammalian IgM.

The Ig monomer is a “Y”-shaped molecule that consists of four polypeptide chains; two identical heavy chains and two identical light chains connected by disulfide bonds between cysteine residues. Each heavy chain is about 440 amino acids long; each light chain is about 220 amino acids long. Heavy and light chains each contain intrachain disulfide bonds which stabilize their folding. Each chain is composed of structural domains called Ig domains. These domains contain about 70-110 amino acids and are classified into different categories (for example, variable or V, and constant or C) according to their size and function. They have a characteristic immunoglobulin fold in which two β sheets create a “sandwich” shape, held together by interactions between conserved cysteines and other charged amino acids.

There are five types of mammalian Ig heavy chain denoted by α, δ, ε, γ, and μ. The type of heavy chain present defines the isotype of antibody; these chains are found in IgA, IgD, IgE, IgG, and IgM antibodies, respectively.

Distinct heavy chains differ in size and composition; α and γ contain approximately 450 amino acids and δ approximately 500 amino acids, while μ and ε have approximately 550 amino acids. Each heavy chain has two regions, the constant region (C_{H}) and the variable region (V_{H}). In one species, the constant region is essentially identical in all antibodies of the same isotype, but differs in antibodies of different isotypes. Heavy chains γ, α and δ have a constant region composed of three tandem Ig domains, and a hinge region for added flexibility; heavy chains μ and ε have a constant region composed of four immunoglobulin domains. The variable region of the heavy chain differs in antibodies produced by different B cells, but is the same for all antibodies produced by a single B cell or B cell clone. The variable region of each heavy chain is approximately 110 amino acids long and is composed of a single Ig domain.

In mammals, there are two types of immunoglobulin light chain denoted by λ and κ. A light chain has two successive domains: one constant domain (CL) and one variable domain (VL). The approximate length of a light chain is 211 to 217 amino acids. Each antibody contains two light chains that are always identical; only one type of light chain, κ or λ, is present per antibody in mammals.

Although the general structure of all antibodies is very similar, the unique property of a given antibody is determined by the variable (V) regions, as detailed above. More specifically, variable loops, three each the light (VL) and three on the heavy (VH) chain, are responsible for binding to the antigen, i.e. for its antigen specificity. These loops are referred to as the Complementarity Determining Regions (CDRs). Because CDRs from both VH and VL domains contribute to the antigen-binding site, it is the combination of the heavy and the light chains, and not either alone, that determines the final antigen specificity.

An “antibody fragment” contains at least one antigen binding fragment as defined above, and exhibits essentially the same function and specificity as the complete antibody of which the fragment is derived from. Limited proteolytic digestion with papain cleaves the Ig prototype into three fragments. Two identical amino terminal fragments, each containing one entire L chain and about half an H chain, are the antigen binding fragments (Fab). The third fragment, similar in size but containing the carboxyl terminal half of both heavy chains with their interchain disulfide bond, is the crystalizable fragment (Fc). The Fc contains carbohydrates, complement-binding, and FcR-binding sites. Limited pepsin digestion yields a single F(ab′)2 fragment containing both Fab pieces and the hinge region, including the H—H interchain disulfide bond. F(ab′)2 is divalent for antigen binding. The disulfide bond of F(ab′)2 may be cleaved in order to obtain Fab′. Moreover, the variable regions of the heavy and light chains can be fused together to form a single chain variable fragment (scFv).

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

### BRIEF DESCRIPTION OF THE FIGURES

Non-limiting, an exemplary embodiment of the disclosure will now be described with reference to the accompanying drawings, in which:

FIG. 1 shows a the dual cartridge drug delivery device in accordance with a first embodiment of the disclosure view with parts removed; and

FIG. 2 in a perspective view a section of the device in FIG. 1 during dose setting;

FIG. 3 a section of figure from another perspective; and

FIG. 4 a distal section of the device in FIG. 1 during dose dispensing.

### DETAILED DESCRIPTION

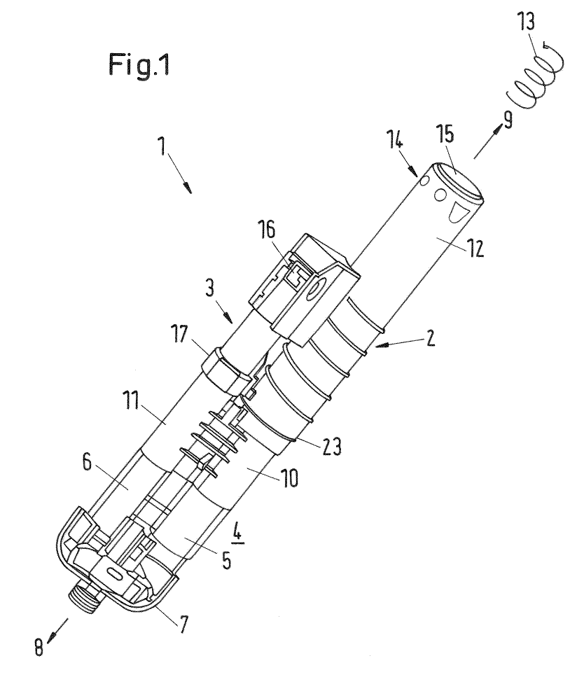

FIG. 1 shows the interior of a drug delivery device 1 comprising a primary drug delivery assembly 2 and a secondary drug delivery assembly 3, both retained in a housing that is only indicated by reference numeral 4. The primary drug delivery assembly 2 serves for the delivery of a primary medicament contained in a primary cartridge 5 attached to the distal end of the primary drug delivery assembly 2. The secondary drug delivery assembly 3 serves for the delivery of a secondary medicament contained in a secondary cartridge 6 attached to the distal end of the secondary drug delivery assembly 3. The cartridges may also be retained in a cartridge holder that is attachable to the distal end of the device 1. The cartridge holders for the cartridges may be integrated in a single cartridge holder hub.

At the distal end of the assembly, a 2-1 needle adapter 7 is attached. At the distal end 8 of the needle adaptor a screw thread is formed. An injection needle can be attached to said screw thread or injection. The injection needle is in fluid communication with two proximally oriented needles in the needle adapter 7 that pierce the respective septa of the respective medicament cartridge 5 and 6. The device 1 extends from the distal end 8 to a proximal end 9.

The primary drug delivery assembly 2 comprises a primary housing, housing body or housing section 10 fixed in the housing 4, and the secondary drug delivery assembly 3 comprises a secondary housing, housing body or housing section 11 fixed in the housing 4. In the primary drug delivery assembly 2 there is a primary dose setting member (not shown) in the form of a dose dial sleeve, which is threadedly engaged with a threaded insert (not shown) fixed in the primary housing section 10 so that when the primary dose setting member is rotated to set an increasing dose of the primary medicament, it moves along a helical pattern in proximal direction. The primary dose setting member is connected to a dose dial 12 via a connection element, which is a dial grip of a regular drug delivery device which is rotationally constrained to the primary dose setting member. The dose dial is configured as a dial collar and assembles as a sleeve over the primary drug delivery assembly 2.

The dose 12 is movable between a first distal axial position and a second proximal axial position. The dose dial 12 is rotationally constrained with respect to the housing 4 when in the first position and when the primary dose setting member is in its most distal position, which corresponds to a set dose of zero units.

When the dose dial 12 is moved proximally in axial direction, it disengages from the rotational lock with the housing so that the dose dial 12 is free to rotate relative to the housing 4. The dose dial 12 is rotationally constrained to the primary dose setting member. The dose dial 12 may be permanently splined to the primary dose setting member by an axially extending spline connection. Rotation of the dose dial 12 is then transferred to the primary dose setting member. The primary dose setting member is usually rotated (in a first rotational direction) to set an increasing dose of the primary medicament. When the dose dial 12 is in the second position, a dose of the primary medicament can be set by rotating the dose dial 12. In this case the dose dial 12 and the primary dose setting member 11 displace in proximal direction in a helical movement.

Inside the primary drug delivery assembly 2, there is a dose dispense mechanism including a clutch, a piston rod and a drive sleeve. For dispense of the primary medicament, a dispense button located in the connection element of the primary drug delivery assembly 2 is pressed. In the embodiment shown, a spring (indicated a 14) urges a dose button 15 in the dose dial 12 in proximal direction away from a dispense button (not shown) of the primary drug delivery assembly 2.

When a dose of a primary medicament has been set, the dose button 15 is pressed so that the dose button acts on the dispense button of the primary drug delivery assembly. The dispense button acts on the clutch mechanism between the primary drive sleeve and the primary dose setting member which is disengaged and the primary dose setting member is forced to move in distal direction in a pure axial motion thereby rotating the piston rod located inside. As the piston rod is in threaded engagement with a housing insert, the piston rod displaces in distal direction thereby displacing a bung in the cartridge 5.

In order to seta dose, the user is forced to follow specific steps. As the dose dial 12 is blocked rotationally within the housing 4 until it is pulled upwards (proximally) from the first position into the second position relative to the primary dose setting member, the user has to pull the dose dial 12 in proximal direction. That causes the engagement with the housing 4 to be removed. Thereafter, the dose dial 12 can be rotated wherein this rotation is transmitted to the primary dose setting member such that a dose of the primary medicament of the primary drug delivery assembly 2 is set.

The secondary drug delivery assembly has inside the second housing body 11 a secondary drive sleeve 16. The secondary drive sleeve 16 surrounds a lead screw (not shown) of the secondary drug delivery assembly 3. The lead screw has two threads. The first thread extends along the lead screw and is in threaded engagement with a thread fixed in the housing 11. The second thread at the proximal end of the lead screw is formed on flexible arms and the inner thread-form of the drive sleeve 16 with which it co-operates is ramped on one side.

When the secondary drive sleeve 16 is moved proximally to set a dose of the secondary medicament to be dispensed, said flexible arms deflect so that the secondary drive sleeve moves proximally relative to the lead screw. The set dose of the secondary medicament is dispensed by moving the secondary drive sleeve 16 axially in distal direction 8 to return it to its starting position. By this action, the thread form of the drive sleeve engages the flexible arm of the lead screw and the lead screw is caused to rotate. Because the lead screw is threaded to housing 11 it advances through this thread to dispense the set dose of the secondary medicament. The lead screw advances by a distance that corresponds to the relationship between the leads of the first and second threads.

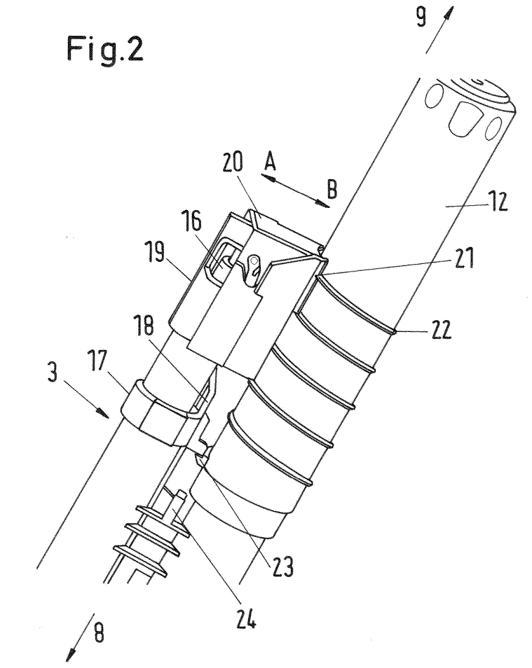

FIG. 2 shows that the secondary drug delivery assembly 3 has a transfer collar 17 with a push arm 18. The transfer collar 17 is moveable in the axial direction between a proximal and a distal position. The arm 18 is connected with the inner section of the secondary housing 11. When the transfer collar 17 is moved proximally, the arm 18 pushed the secondary drive sleeve 16 in proximal direction via a secondary dose setting member (not shown) which can be integrally formed with the secondary drive sleeve 16. By moving the secondary drive sleeve 16 in proximal direction, a dose of the secondary medicament is set. The possible displacement of the transfer collar 17 in proximal direction limited by a housing stop (not shown) engaging the arm 18. Thereby, the set dose of the secondary medicament is a fixed dose.

At the proximal end of the secondary drug delivery assembly, there is a dispense actuator 19 which urges the secondary drive sleeve in distal direction when moved distally. A box-shaped clutch component 20 is located at the proximal end of the secondary drug delivery assembly and accommodates a section of the dispense actuator 19. Toward the dose dial 12, a section of the clutch component 20 is provided with a thread interface, here an axially extending thread section 21. The dose dial has a corresponding thread 22 on an outer surface. The clutch component is moveable in a direction perpendicular to an axis running from the proximal end 9 to the distal end 8 between a first position A and a second position B. In the first position the thread interface 21/22 is not engaged. When the clutch component is moved into the second position B, then rotation of the dose dial 12 causes the outer thread 22 to engage the thread 21. The engagement can be the result of the displacement of the clutch component 20 towards the dose dial (direct engagement) or the result of a following rotation of the dose dial (indirect engagement).

At a distal section of the dose dial 12, the dose dial 12 has an arm 23. When the dose dial is in the most distal position, the arm 23 engages a flexible rib 24 on the housing. The engagement between the arm 23 and the rib 24 locks the dose dial 12 against rotation. To disengage the arm 23 from the rib 24, the user has to pull the dose dial 12 in proximal direction 9. When the user pulls the dose dial 12, the arm 23 engages the transfer collar 17 (see FIG. 1) and moves it in proximal direction such that a dose of the secondary medicament is set. This pure axial motion of the dial is further secured by a splined connection between the dose dial 12 and the housing 10 (not shown). This splined connection locks the dose dial 12 until the dose dial 12 reaches the proximal second position, where the dose dial is free to rotate and where the dose dial is rotationally locked to the primary dose setting member. The arm 23 moves the transfer collar 17 proximally until the dose dial 12 reaches the second position.

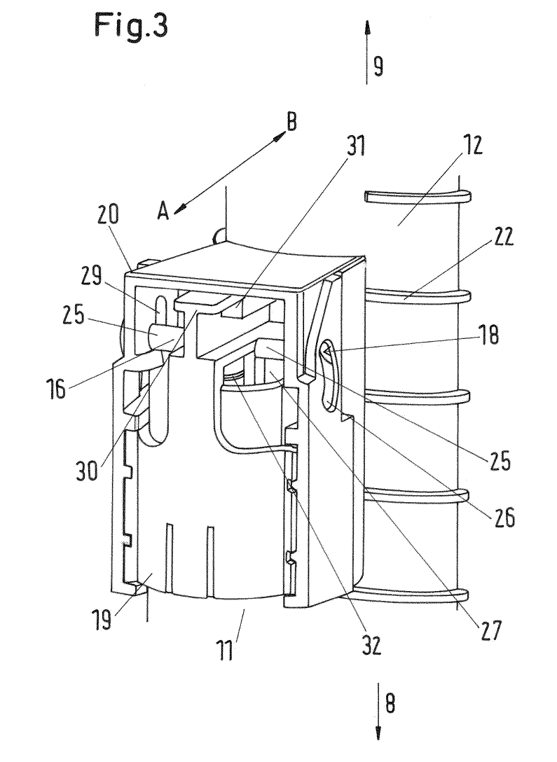

As shown in FIG. 3, the secondary drive sleeve 16 has flexible arms 25 which engage a first track 26 provided on the clutch component 20. The arms 25 extend outwardly. At the lower side of the arms 25 there is a slanted surface 27. When the secondary drive sleeve 16 moves distally, the slanted surface interacts with the housing body 11 and the arms deflect inwardly. Pins 28 formed on the arms 25 are thereby moved from the first track 26 into a second track 29 formed on an inner surface of the clutch component 20.

The clutch component 20 is guided laterally on the dispense actuator 19, which as a horizontally extending bar 30 engaging a recess 31 on the clutch component 20. Further nut/groove connections between the clutch component 20 and the dispense actuator 19 are located on a lower side. The dispense actuator is movable relative to the housing body 11 in the axial direction.

When the secondary drive sleeve 16 is moved proximally when the dose dial 12 is moved in proximal direction 9, the pins 28 engage the first track 26, which is curved. This shape of the track 26 forces the clutch component 20 to move from the first position A to the second position B in horizontal direction and into engagement with the dose dial 12. The dose dial 12 is now in the second position and ready to be rotated to set an increasing dose of the primary medicament beginning with zero units of the primary medicament. Rotation of the dose dial 12 does not cause displacement of the secondary drive sleeve 16 so that there is no further dose setting in the secondary drug delivery assembly 3.

For dispense, the user depresses the dose button 15. The depression of the dose button 15 displaces the dose dial 12 and by means of the thread engagement with the clutch component, also the clutch component. During this phase, the dose dial 12 is locked against rotation as the primary dose setting member is locked against rotation. The spring 14 that urges the dose button 15 in proximal direction applies a force that is stronger than a force that is required to displace the secondary drive sleeve 16.

The distal displacement of the clutch component 20 is transferred to the dispense actuator 19, which urges the secondary drive sleeve 16 in distal direction so that the set dose of the secondary medicament is dispensed. At the end of the distal travel of the secondary drive sleeve 16, the arms 25 deflect inwardly and the pin 28 moves into the second track 29. Dispense of the secondary medicament is now finished. When the pin 28 is in the second track, the secondary drive sleeve moves a small distance in proximal direction under the force of the spring 32. The second track 29 is formed such that the clutch component 20 is moved away from the dose dial from the position B to the position A, thereby disengaging the thread of the clutch component 20 from the thread of the dose dial 12.

After dispense of the secondary medicament, the dose button 15 reaches the primary dispense button. The dose button 15 engages the primary dispense button and initiates dispense of the primary medicament by declutching the clutch inside the primary drug delivery assembly 2. Further displacement of the dose dial 12 by depressing the dose button 15 causes the primary dose setting member 11 to rotate back in distal direction thereby dispensing the primary medicament. Accordingly, the fixed dose of the secondary medicament is dispensed prior to the variable dose of the primary medicament.

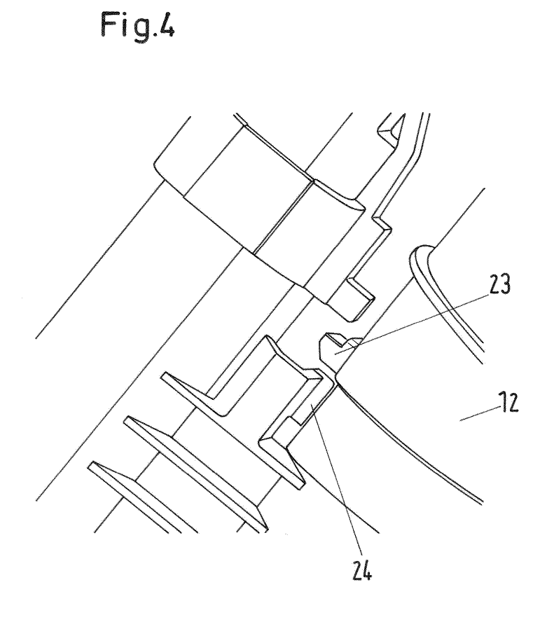

At the end of the dispensing process, the arm 23 (see FIG. 4) of the dose dial 12 reengages with the flexible rib 26. If the dose dial 12 has been set from the first into the second position at the beginning of the dose setting process and the dose dial 12 has not been rotated yet, the flexing rib 26 prevents the dispensing mechanism (dose dial 12) from being be displaced distally. Thus, before a set dose of the secondary medicament can be dispensed, the user is required to set a dose of the primary medicament. In turn, as the user is required to pull the dose dial 12 before he is able to set a dose of the primary medicament, the user always receives a fixed dose of the secondary medicament in combination with a variable dose of the primary medicament.

### REFERENCE NUMERALS

1 drug delivery device
2 primary drug delivery assembly
3 secondary drug delivery assembly
4 housing
5 primary cartridge
6 secondary cartridge
7 needle adapter
8 distal end
9 proximal end
10 primary housing
11 secondary housing
12 dose dial
13 helical movement
14 spring
15 dose button
16 secondary drive sleeve
17 transfer collar
18 arm
19 dispense actuator
20 clutch component
21 thread
22 outer thread
23 arm
24 flexible rib
25 flexible arm
26 first track
27 slanted surface
28 pin
29 second track
30 bar
31 recess
32 spring

1-13. (canceled)

14. A drug delivery device comprising:
a housing retaining a primary drug delivery assembly for the delivery of a primary medicament, wherein the primary drug delivery assembly comprises:
a primary dose setting member configured to rotate in a helical movement in a proximal direction to set a dose of the primary medicament; and
a secondary drug delivery assembly for the delivery of a secondary medicament,
the drug delivery device further comprising a dose dial movable between a first position and a second position in an axial direction with respect to the primary dose setting member,
wherein the dose dial is rotationally constrained with respect to the housing by an axially splined connection to the housing when moved from the first position into the second position when the primary dose setting member is in a zero unit dose position,
wherein the dose dial is rotationally constrained to the primary dose setting member in the second position by spline features that engage corresponding spline features on the primary dose setting member in the second position and is free to rotate relative to the housing so that a dose of the primary medicament is set,
wherein the secondary drug delivery assembly comprises a clutch component having a thread interface configured for engagement with a thread interface provided on the dose dial and coupled to a dispense actuator of the secondary drug delivery assembly,
wherein a dose button is arranged in the dose dial in such way that displacement of the dose button in a distal direction causes the dose dial to move in the distal direction from the second position into the first position relative to the primary dose setting member,
wherein the drug delivery device is configured such that when the dose dial is moved from the first position into the second position, the clutch component is moved from a first position, where the clutch component is disengaged from the dose dial, to a second position, where the clutch component engages the dose dial so that during rotation of the dose dial in the second position, the thread interface of the dose dial and the thread interface of the clutch component engage such that the dose dial moves in the axial direction relative to the clutch component and such that pure axial motion of the dose dial in the distal direction from the second position into the first position by the dose button is transferred to the dispense actuator such that a fixed dose of the secondary medicament is dispensed, and
wherein the secondary drug delivery assembly comprises a secondary dose setting member and a transfer collar, wherein the secondary dose setting member is configured to move in the proximal direction, wherein the dose dial is configured to engage the transfer collar when moved from the first position into the second position, wherein the transfer collar transfers a proximal movement of the dose dial to the secondary dose setting member such that the fixed dose of the secondary medicament is set.

15. The drug delivery device according to claim 14, wherein the clutch component is slidably guided on the dispense actuator between the first position, where the clutch component is disengaged from the dose dial, and the second position, where the clutch component engages the dose dial.

16. The drug delivery device according to claim 14, wherein the secondary drug delivery assembly comprises a secondary drive sleeve and a lead screw, wherein the secondary drive sleeve is coupled to the secondary dose setting member and is configured to move in the proximal direction to set a dose of the secondary medicament and is configured to move in the distal direction during dispensing to drive the lead screw in the distal direction.

17. The drug delivery device according to claim 16, wherein the dispense actuator is configured to move to the secondary drive sleeve in the distal direction when actuated, and wherein the secondary drive sleeve is configured to drive the lead screw for dispensing the secondary medicament when moved in the distal direction.

18. The drug delivery device according to claim 16, wherein the secondary drive sleeve comprises pins engaging a first track on the clutch component, wherein the first track is configured such that movement of the pins in the first track causes the clutch component to engage the dose dial when a dose of the secondary medicament is set.

19. The drug delivery device according to claim 18, wherein the secondary drive sleeve comprises flexible arms, wherein the pins are provided on the flexible arms, wherein the flexible arms are configured to deflect from the first track when the secondary drive sleeve is moved in the distal direction to engage a second track on the clutch component, and wherein the second track is configured such that an axial displacement of the secondary drive sleeve moves the clutch component into the first position.

20. The drug delivery device according to claim 16, wherein the secondary drug delivery assembly comprises a reset spring arranged to bias the secondary drive sleeve in the axial direction after dispensing the secondary medicament.

21. The drug delivery device according to claim 14, wherein when the dose dial is in the first position, the dose button engages a dispense button of the primary drug delivery assembly such that further movement of the dose button in the distal direction causes a set dose of the primary medicament to be dispensed.

22. The drug delivery device according to claim 14, wherein the dose dial is splined to a rib provided in the housing until the dose dial has been moved from the first position into the second position.

23. The drug delivery device according to claim 22, wherein the primary dose setting member is configured to move in the distal direction in a helical movement during dispensing the primary medicament, wherein the dose dial is configured to reengage with the rib after dispensing the primary medicament.

24. The drug delivery device according to claim 14, comprising a primary medicament cartridge and a secondary medicament cartridge, wherein the primary medicament cartridge is coupled to the primary drug delivery assembly and the secondary medicament cartridge is coupled to the secondary drug delivery assembly, and wherein the primary medicament cartridge contains the primary medicament and the secondary medicament cartridge contains the secondary medicament.

25. The drug delivery device according to claim 14, wherein the drug delivery device is configured as a disposable device.

26. The drug delivery device according to claim 14, wherein the dose dial is configured as a sleeve that at least partly surrounds the primary dose setting member.

27. The drug delivery device according to claim 24, wherein at least one of the primary medicament and the secondary medicament comprises a pharmaceutically active compound.
